(19) United States
(12) Patent Application Publication
(10) Pub. No.: US 2007/0184020 A1
(43) Pub. Date: Aug. 9, 2007

(54) COMBINATION OF RADIO WAVES WITH PHARMACOLOGICALLY ACTIVE SUBSTANCES

(75) Inventors: Jochen Kalbe, Leichlingen (DE); Georg Ludwig, Eschen (LI)

Correspondence Address: AMIN, TUROCY & CALVIN, LLP, 1900 EAST 9TH STREET, NATIONAL CITY CENTER, 24TH FLOOR,, CLEVELAND, OH, 44114, US

(73) Assignees: Jochen Kalbe, Leichlingen (DE); Georg Ludwig, Eschen (LI)

(21) Appl. No.: 11/570,206

(22) PCT Filed: Jun. 9, 2005
(86) PCT No.: PCT/DE05/01028

§ 371 Date: Dec. 7, 2006

(30) Foreign Application Priority Data
Jun. 9, 2004 (DE) 102004028156.4

(51) Int. Cl. A61N 1/39; A61K 38/19; A61K 31/7076; A61K 31/7072; A61K 31/7048; A61K 31/704

(52) U.S. Cl. 424/85.1; 514/44; 514/34; 514/8; 514/27; 514/183; 514/171; 514/47; 514/49; 514/575; 514/449; 514/263.31; 514/269; 514/109; 424/649; 607/86; 607/1

(57) ABSTRACT

The invention relates to a combination of radio waves with pharmacologically active substances chosen from the group: a) monoclonal antibodies and/or b) tyrosine-kinase inhibitors and/or c) angiogenesis inhibitors and/or d) farnesyl-transferase inhibitors and/or e) topoisomerase-I or -II inhibitors and/or f) cytokines and/or g) antisense oligonucleotides. optionally together with one or more chemotherapeutics, as well as to the use of this combination for the prophylaxis and/or treatment of cancer, tumors and metastases.

The invention relates to a combination of radio waves with pharmacologically active substances. Moreover, the inventive combination can comprise chemotherapeutics and is preferably used for prophylaxis and/or treatment of tumors and metastases.

The therapy of cancer with different radiation methods has been known for a long time and is state of the art. Inter alia, radiation with x-rays, gamma radiation and with lower energy radiation such as microwaves are included herein.

Besides radiation with high energy x-rays or radioactive radiation there are alternative techniques for treating tissue with electromagnetic radiation:
1. microwave, frequency range from 400 to 2500 MHz
2. ultrasound, frequency range from 0.5 to 5 MHz
3. radio frequency, frequency range from 3 to 300 MHz
4. “hot source”-hyperthermia, hot water
5. laser

Radiation with radio waves is especially suitable for local treatment and is performed in Europe according to the directives of the ESHO (European Society of Hyperthermic Oncology) (Hand, J. W. Quality Assurance Guidelines for ESHO protocols, Int. J. Hyperthermia 5, 1989, 421-428).

Combinations of different radiation therapies with hyperthermia, preferably generated by microwave treatment, are known and researched (Hehr, T., Current and Potential Role of Thermoradiotherapy for Solid Tumors, Onkologie, 2003, 26, 295-302; Seegenschmiedt M. H., Radiotherapie und Hyperthermie—ein neuer Weg in der interdisziplinären Tumortherapie, Jahrbuch Radiologie (Günther ed), Springer, 161-171, 1994). The synergies of this combination therapy are currently being examined and are not yet proven sufficient (van der Zee J, Point-counterpoint: what is the optimal trial design to test hyperthermia for carcinoma of the cervix? Point: addition of hyperthermia or cisplatin to radiotherapy for patients with cervical cancer; two promising combinations—no definite conclusions, International Journal of Hyperthermia, 2002, Vol. 18 (1), 19-24).

The combination of hyperthermia with chemotherapeutics is also a therapy method already in use (WO 93/18751, Liposome encapsulated taxol and method of using the same; Kuwano H., Preoperative hyperthermia combined with chemotherapy and irradiation for the treatment of patients with esophageal carcinoma, Tumori, 1995, Vol. 81 (1), 18-22; Rietbroek R C, Hyperthermia in combination with chemotherapy in gynecological cancers, Nederlands tijdschrift voor geneeskunde, 2002, Vol. 143 (2), p: 85-8; Colombo R., Multicentric study comparing intravesical chemotherapy alone and with local microwave hyperthermia for prophylaxis of recurrence of superficial transitional cell carcinoma, Journal of Clinical Oncology, 2003, Vol. 21 (23), 4270-6).

In chemotherapy treatment several positive effects are in principle postulated by means of the additional treatment with hyperthermia: On the one hand, an improved bioavailability of the chemotherapeutics in the tumor through an enhanced transport within and to the tumor, and on the other hand a higher effectiveness in the already thermally loaded and hence weakened tumor.

However, depending on the tumor and on the chemotherapeutic, different effects can occur, which also show that the additional treatment with hyperthermia provides no benefit (van Bree C., Local hyperthermic treatment does not enhance mitoxantrone effectiveness for responses of a rat solid tumor regrowing after irradiation, Journal of Cancer Research and Clinical Oncology, 1996, 122 (3), 147-53).

The object of the present invention is to provide a combination of electromagnetic radiation and substances, which is suitable for the prophylaxis and/or treatment of cancer, tumors and metastases.

This object is solved by the technical teaching of the independent claims. Further advantageous embodiments, aspects and details of the invention result from the dependent claims, the description and the examples.

Surprisingly it was found, that a combination of radio waves and certain substances is very well suited for the prophylaxis and treatment of cancer, tumors and metastases.

In the case of the aforementioned substances at least one pharmacologically active substance is preferred and further, pharmacologically active substances used in cancer therapy are preferred.

In particular, in the case of these preferred substances, the active agents are chosen from the group comprising:
a) monoclonal antibodies and/or
b) tyrosine-kinase inhibitors and/or
c) angiogenesis inhibitors and/or
d) farnesyl-transferase inhibitors and/or
e) topoisomerase-I or -II inhibitors and/or
f) cytokines and/or
g) antisense oligonucleotides.

Therapeutic antibodies to be used according to invention are monoclonal antibodies against tumor cells and metastases. Included herein are antibodies such as cetuximab, trastuzumab, alemtuzumab, rituximab, bevacizumab, EMD7200, rencarex, HeFi-1, apolizumab, tositumomab, ABX-EGF, HuMAx-EGFR, labetuzumab, pemtumomab, triab, Mab17-1A (panorex), MDX-210, MDX-220, MDX-447, MDX-H210, gemtuzumab ozogamicin, radio-marked antibodies such as Indium-111 (In-111) ibritumomab tiuxetan, yttrium-90 (Y-90) ibritumomab tiuxetan, Anti-CD80 mAb, WX-G250RIT (I-131).

Tyrosine-kinase inhibitors are molecules which prevent the function of tyrosine-kinase. This can occur through blocking of the epidermal growth factor receptor (EGFR) such as for example ErB-1, ErB-2 (Her2/Neu), ErB-3, ErB-4, or of other receptors of transmembrane factors such as vascular endothelium growth factor (VEGF), fibroblast growth factor (FGF), platelet derived growth factor (PDGF), insulin like growth factor (IGF-I) or hepatocyte growth factor (c-met). Herein, antibodies as well as “small molecules” can be concerned. Examples of “small molecules” to be used according to the invention are gefitinib, imatinib, PKI-166, CI-1033, and SU-6668.

Angiogenesis inhibitors are substances which suppress the growth of the blood supplying vessels of the tumor. Included herein are for example cilengitide, neovastat, IM862, SU5415, SU5416, SU6668 and suramin.

Farnesyl-transferase inhibitors are for example BMS214662 and R115777 (zarnesta tipifarnib).

Examples for cytokines as immunomodulators are aldesleukin, interleukin-2, interleukin-12, interferons such as interferon-alpha 2a, interferon-alpha 2b, interferon beta, interferon gamma and recombinant tumor necrosis factors such as tasonermin.

Included in the antisense oligonucleotides is for example AP12009.

Chemotherapeutics to be used according to invention are folic acid antagonists such as methotrexate, antagonists of purine and pyrimidine bases such as fluorouracil, capecitabine, gemcitabine, cytarabine, pentostatin, cytosine arabinoside, mercaptopurine, fludarabine, cladribine, thioguanine, paclitaxel, nitrourea, etoposide, campathecin, bleomycin, pliamycin, mitoxantron, cytochalasin B, gramicidin B, ethidiumbromide, emetin, mitomycin, tenoposides, colchicine, mithramycine, 1-dehydrotestosterone, glucocorticoides, procaine tetracaine, lidocaine, propanolol, puromycine, streptozoticine, alkylating cytostatics such as cyclophosphamide, ifosfamide, melphalan, thiotepa, busulfan, cisplatin, carboplatin, oxaliplatin, procarbazine, dacarbazine, temozolomide, etoposide, teniposide, mitosis inhibiting agents such as vinblastine, vincristine, vindesine, vinorelbine, paclitaxel, docetaxel, trofosfamide, chlorambucil, treosulfan, estramustine, nimustine, carmustine, lomustine, cytostatically active antibiotics such as dactinomycin, anthracyclines such as daunorubicin, doxorubicin (adriamycin), doxorubicin lipo, idarubicin and epirubicin, bleomycine, mitomycines, mitoxantrone, amsacrine, actinomycin D, hormones and hormone antagonists such as buselerine, goselerine, leuproreline, triptoreline, anti-estrogens such as tamoxifen, aromatase inhibitors such as aminoglutethimide, anastrozole, letrozole, exemestane, formestane, testolactone, glucocorticoids, other cytostatics such as miltefosine, hydroxyurea, tretinoin, topoisomerase inhibitors such as camptothecin derivatives such as irinotecan or hycamtin, topotecan or other chemotherapeutics such as for example imatinib, amsacrine, pentostatin, bexaroten and asparaginase.

The aforementioned chemotherapeutics can be used optionally together with the combination according to the invention.

Surprisingly it was also found, that radio waves (3 to 300 MHz) significantly increase the effectiveness of certain substances for fighting cancer, tumors and metastases. This occurs in many cases with simultaneous reduction of side effects of these substances. As substances can be used the above mentioned pharmacologically active substances and more preferred are the pharmacologically active substances used in cancer therapy as well as the chemotherapeutics.

Thus the present invention is also directed to the use of radio waves for increasing the activity of the aforementioned substances and/or the reduction of the side effects of the aforementioned substances.

By the term “increasing the activity”, for example, the reduction of the IC₅₀-value of a pharmacologically active substance is understood. An increase of the activity means also that approximately the same result is achieved with substances having less pharmacologic activity.

The reduction of side effects preferably relates to the chemotherapeutics (cytostatics) and the side effects caused by these active agents, respectively. The side effects of chemotherapeutics are well known to the person skilled in the art and are serious problems in tumor therapy.

The possibility of a successful therapy is significantly higher when the combination according to invention is used and the patients' quality of life also is significantly improved. Further, it is important that the treatment with radio waves is effected in a non-invasive form, i.e., that the electrohyperthermia uses electrodes, which require no surgical intervention and which are placed on the skin in a minimum distance to the tumor and directly radiate the tumor through the skin. Thus, it is not necessary to directly place the electrodes in the tumor region for example by using a catheter. Thus, the cost of the treatment is reduced and the treatment is considerably more comfortable and less dangerous for the patient. The combination according to invention is used especially in the case of the following cancer, or respectively tumor and metastase types: breast carcinomas, especially recurrent tumors after radiation and chemotherapy, skin carcinomas such as basaliomas, spinaliomas, melanomas, skin metastases, all malignant tumors which are located close to the body surface, soft tissue tumors, endometrial carcinoma, cervical carcinoma, ovarian cancer, ovarian carcinoma, testicular carcinoma or respectively vulva carcinoma, germ cell tumors, prostate cancer, thyroid carcinomas, liver cell carcinomas, liver metastases, colon cancer or respectively colorectal carcinoma, rectal carcinoma, lung cancer such as small cell and non-small cell bronchial carcinoma, lymph node cancer, Hodgkin's and Non-Hodgkin's lymphomas, pancreas cancer or respectively pancreatic carcinoma, connective tissue tumor, soft tissue sarcoma, adenocarcinomas such as stomach, pancreas, gallbladder, esophagus carcinomas, other stomach carcinomas, osteolytic carcinomas and osteoplastic carcinomas, renal cell carcinomas, malignomas of the gastrointestinal tract, brain tumors such as glioblastomas, astrocytomas, tumors of the throat, nose and ear area, malignant neoplasma, neuroblastoma, choroidal melanoma, acute leukemia, acoustic neurinoma, ampullary carcinoma, anal carcinoma, bladder cancer, breast cancer, Burkitt's lymphoma, corpus cancer, CUP-syndrome, cancer of the small intestine, tumors of the small intestine, ependymoma, epithelial cancer types, Ewing's tumors, gastrointestinal tumors, gallbladder cancer, uterine cancer, cervical cancer, gynecologic tumors, hematologic neoplasias, hairy cell leukemia, urethral cancer, skin cancer, brain metastases, hypophysis tumor, carcinoids, Kaposi's sarcoma, laryngeal cancer, bone cancer, colon carcinoma, craniopharyngiomas, cancer in the mouth area and on lips, leukemia, eyelid tumor, lymphomas, rectum cancer, medulloblastomas, melanomas, meningeomas, Hodgkin's disease, mycosis fungoides, nose cancer, neurinoma, renal cancer, oligodendroglioma, esophageal carcinoma, osteosarcoma, penile cancer, plasmocytoma, esophageal cancer, retinoblastoma, vaginal cancer, Schneeberger's disease, T-cell lymphoma (mycosis fungoides), thymoma, tube carcinoma, eye tumors, urethral cancer, urologic tumors, urothelial carcinoma, Wilm's tumor and tongue cancer.

In the case of the combination according to invention the radio waves are in a frequency range from 3 to 30 MHz, preferred from 5 to 20 MHz and especially preferred from 10 to 15 MHz. Especially preferred is a frequency of 13.56 MHz. Further the radio waves are preferably generated by capacitive coupling.

Moreover the present invention relates to a method for the prophylaxis and/or treatment of cancer, tumors and/or metastases by application of the combination according to the invention. The tumors are preferably cancerous and benign tumors.

In this method for the prophylaxis and/or treatment of cancer, tumors and/or metastases, radio waves are used in combination with at least one active agent chosen from the group:
a) monoclonal antibodies and/or
b) tyrosine-kinase inhibitors and/or
c) angiogenesis inhibitors and/or
d) farnesyl-transferase inhibitors and/or
e) topoisomerase-I or -II inhibitors and/or
f) cytokines and/or
g) antisense oligonucleotides.

Therein, the radio wave field is preferably applied extra-tumorally.

As already mentioned, one or more chemotherapeutics can be applied additionally to the administration of one or more of the active agents mentioned under a)-g).

The chemotherapeutics are preferably chosen from the above mentioned group. In the case of the active agents mentioned under a)-g), also the aforementioned active agents come into question.

The radio wave field is generated preferably through capacitive coupling and is applied preferably extra-tumorally. The treatment with radio waves is carried out with devices which generate the radio wave field by capacitive coupling. Capacitive coupling is based on the tissue being located between two electrodes, which are fed through an alternating current power supply source and serve as capacitor plates. The electromagnetic field builds up between these electrodes in the form of a medium wave radio wave.

The radio wave field is applied preferably extra-tumorally, especially preferred extra-tumorally and extra-corporeally. That means that generally no invasive interventions are necessary for the treatment with radio waves, whereas in the case of tumors which are difficult to access also electrodes to be applied invasively can be introduced into the region of the tumor. The method according to the invention for the treatment of cancer, tumors and/or metastases thus uses an extracorporeal source for radio waves which is combined with the administration of at least one of the antitumor substances described herein. Thus, the present invention discloses the use of at least one source for radio waves, which is preferably extratumoral or extracorporeal, in combination with at least one anticancer substance for preparing a combination which is suitable. for the treatment of cancer, tumors and/or metastases. The at least one antitumor substance is normally administered by inhalation or by intravenous, intraperitoneal, intramuscular, subcutaneous, mucocutaneous, oral, rectal, transdermal, topical, buccal, intradermal, intravaginal, intragastral, intracutaneous, percutaneous, or sublingual administration.

The frequencies of the radio waves or the radio wave field, respectively, are within the range from 3 to 30 MHz, preferred from 5 to 20 MHz, especially preferred from 10 to 15 MHz and in particular preferred at 13.56 MHz, i.e. at the frequency which is approved by the telecommunications office in Europe for such applications.

In the case of the described method, the radiation with radio waves and the application of the active agent can be effected simultaneously or at different times, with 72 hours, preferably 48 hours and especially preferably less than 24 hours between the administration of the active agent and the treatment with radio waves.

Depending on the indication it is preferred that the treatment with radio waves is effected simultaneously with the administration of the active agent or at different times than the administration of the active agent. The preferred time intervals for the delayed application of radiation and administration of the active agent are 12, 24, 36, 48, 60 and 72 hours, wherein 24, 48 and 72 hours are especially preferred. For the one skilled in the art, only a few experiments are necessary for finding out the best sequence of radiation and administration of the at least one active agent, possibly in combination with at least one chemotherapeutic.

Also the at least one active agent mentioned under a)-g) can be further administered simultaneously with the at least one chemotherapeutic or can be administered at another time than said chemotherapeutic. The time difference can be between the fraction of an hour up to a few days. However, simultaneous administration is preferred.

The active agents and chemotherapeutics are applied in usual formulations and preferably systematically.

### DESCRIPTION OF THE FIGURES

FIG. 1 shows the course of the treatment during 90 days of the treatment of carcinomas of the large intestines by means of cetuximab, as well as by means of cetuximab in combination with electrohyperthermia (EHT), (CEA and CA 19-9 are tumor markers).

FIG. 2 shows the course of the treatment during several months of a therapy of pancreatic carcinomas with liver metastasis by means of a combination of Gemzar and Cetuximab as well as local electrohyperthermia (EHT).

### EXAMPLES

### Example 1

Electrohyperthermia:

Hyperthermia for the treatment of cancer means selectively heating up the carcinogenic tissue for killing cancer cells. Herein, the temperature during the treatment is an important parameter. The cells however protect themselves by means of so-called heat-shock proteins (HSP) from thermal destruction. In electrohyperthermia, the target tissue is heated up by means of electricity, which leads to a reduced synthesis of heat-shock proteins in the cells in comparison to the normal hyperthermia process and thus, the destruction of tumor cells is substantially more efficient.

In the electrohyperthermia, an apparatus having an operating frequency of 13.56 MHz which is exactly adjusted thereto which has following specification:
Principle of operation: Application of radio frequencies with capacitive coupling
Method: Electrohypethermia or respectively loco-regional electrohyperthermia
Radiated power in the range of 30-150 W
Transmitter: water-bolus
Control parameters of the treatment: equivalent temperature
   A Possible Principle of the Activity of Electrohyperthermia:

An electric field which is externally applied can maintain temperature gradients of 1 K/m and thus generate a permanent heat flow of 1500 nW/M² which is clearly more than the natural heat flow (20 nW/m²) through the target cell membranes. This gradient and the resulting heat flow can lead to currents of 150 pA/m² through the membrane, at first due to the fact that Na⁺ flows into the cell; these flows are significantly more than the typically available sodium flow rate of 123 pA/m² from the cell. Thus, the membrane is depolarised and destabilised and the Na⁺/K⁺ pump is reinforced. For this purpose, ATP is required, which further increases the generation of heat on the membrane. The membrane is considerably more permeable to water than to ions, therefore, water is the compound which is most transported in the thermodynamic coupling. A thermal flow of 0.001 K/nm can thus build up a pressure of up to 1.32 MPa. Since malign cells in general have relatively rigid membranes due to the higher concentration of phospholipids, an increased pressure selectively destroys the malign cells before affecting the healthy cells. This principle of activity is not yet verified from the scientific point of view and therefore, is to be considered as hypothetic.

During the treatment, an automatic synchronisation (standing-wave-ratio; SWR<1.1) provides for a standing electric result. As a consequence of the relatively low field intensity (max. 500 V/m) which is applied between the electrodes and the capacitor (applicator for coupling at the region of the body to be treated), a reduced penetration (about 10⁶ V/m) into the interior of the cell which is protected by membranes results therefrom.

The equipment developed for electrohyperthermia comprises an applicator in which the patient is the dielectric in the capacitor and thus, is part of the electric resonance circuit which is exactly adapted and hence, a highly precise adjustment of the standing wave ratio is possible.

The electrohyperthermia device can do the adaptation and synchronization as well as all fine adjustments normally to be carried out by operators and regularly measures the electric parameters for always controlling the process. For measuring the temperature of the treated tissue, the measured absorbed energy as well as the impedance are used. The adaptation as well as the adjustment of the applicator is done based on electrodynamical calculations. A relatively low total energy can be used for the treatment due to the good selectivity as well as the heat absorption which is precisely adjusted.

Further, the surface of the capacitor, which serves for the capacitive coupling to the patient, is well cooled for avoiding burns of the skin and for making possible to apply higher energy during the treatment without risking at the same time to overheat the patient. Normally, the heat energy is not sufficient for heating up the skin to more than 40° C.

The patients are treated by approaching the electrode(s) as close as possible to the surface of the skin and in that the distance of said electrode(s) to the tumor is as small as possible. The treatment period is one hour on three days per week. It is substantial that neither during nor after the treatment, any side effects can be observed.

### Example 2

Treatment of carcinomas of the large intestine with cetuximab, as well as with cetuximab in combination with electrohyperthermia.

4. Patients with a metastatic colorectal carcinoma (mCRC) in a comparable stage (CEA ˜6000 ng/ml) were divided into two groups.

Each group was treated with the monoclonal antibody cetixumab (400 mg/m² initial dosage, 250 mg/M² weekly dosage) and 2 of the 4 patients were additionally treated with local electrohyperthermia on three days per week (Monday, Wednesday and Friday) for respectively one hour. The treatment was continued during a period of 10 weeks.

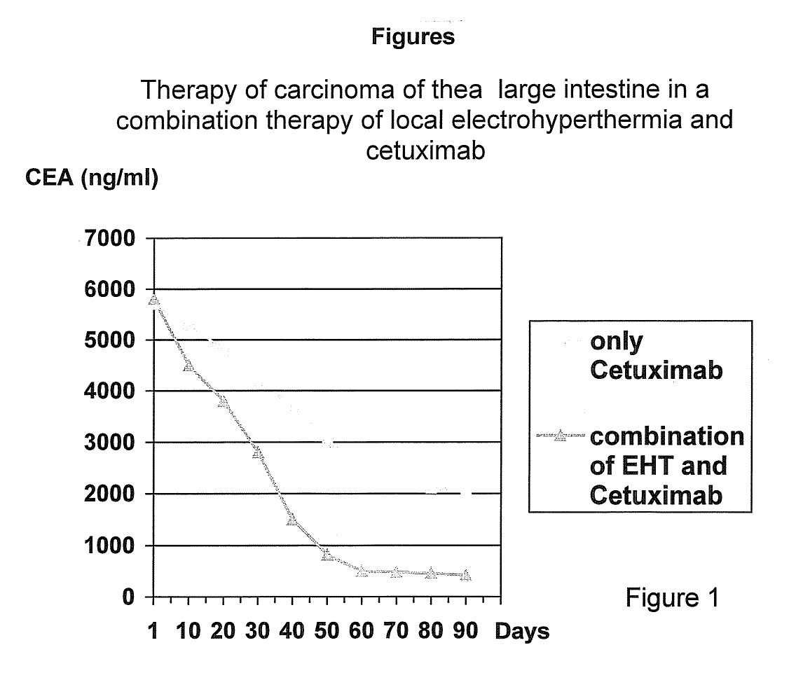

FIG. 1 clearly shows that the CEA was significantly lower after 60 days already in the case of the patients which were additionally treated with electrohyperthermia than in patients which only were treated with the cetuximab antibody.

### Example 3

Therapy of pancreatic carcinomas with liver metastasis by means of a combination of gemzar and cetuximab as well as local electrohyperthermia.

A patient suffering from metastatic pancreatic carcinoma was treated with a combination of Gemzar (1000 mg/M²) and Cetuximab (400 mg/m² initial dosage, 250 mg/M² weekly dosage) during a period of 4 months. During this treatment, the level of the tumor markers CEA and CA 19-9 continuously increased. In the eighth month, the patient was additionally treated with electrohyperthermia. The additional treatment with electrohyperthermia was carried out on three days per week for respectively one hour. The administered dosage of gemzar and cetuximab was not altered during this period.

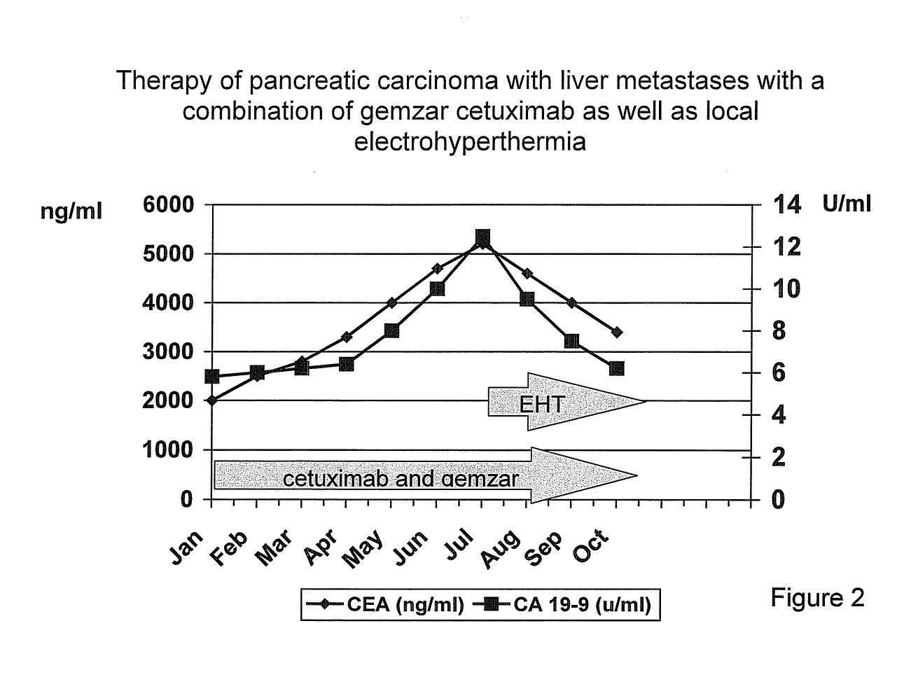

FIG. 2 clearly shows that the values of both tumor markers significantly decreased as soon as the additional treatment with local electrohyperthermia had started.

### Further Examples

Tests according to example 2 are being performed until now for the following tumors:

Cervical tumor, non-Hodgkin carcinoma, melanoma, rectal carcinoma, tumor of the throat and the neck, sarcoma of the soft tissue, lung cancer, bladder cancer, breast cancer, recurring breast cancer, small-cell lung carcinoma, glioblastoma, stomach cancer and esophagus cancer.

As active agents which can be additionally administered with the electrohyperthermia treatment, the following substances can be used:

Vincristine, doxorubicin, epirubicin, imatinib, rencarex, cilengitide, neovastat, interferon gamma, dacarbazin, cyclophosphamide, daunorubicin and interleukin.

First results which can be seen in FIG. 1 are positive.

It is to be noted that the electrohyperthermia is applied as a non-invasive treatment method and the electric field is applied extracorporeally, such that an easy treatment, which is comfortable and gentle for the patient, can be achieved.

1. Combination of radio waves with at least one active agent chosen from the group comprising
a) monoclonal antibodies and/or
b) tyrosine-kinase inhibitors and/or
c) angiogenesis inhibitors and/or
d) farnesyl-transferase inhibitors and/or
e) topoisomerase-I or -II inhibitors and/or
f) cytokines and/or
g) antisense oligonucleotides.

2. Combination according to claim 1, further comprising at least one chemotherapeutic.

3. Combination according to claim 2, wherein the at least one chemotherapeutic are folic acid antagonists, methotrexate, antagonists of purine and pyrimidine bases, fluorouracil, capecitabine, gemcitabine, cytarabine, pentostatin, cytosine arabinoside, mercaptopurine, fludarabine, cladribine and thioguanine, alkylating cytostatics, cyclophosphamide, ifosfamide, melphalan, thiotepa, busulfan, cisplatin, carboplatin, oxaliplatin, procarbazine, dacarbazine, temozolomide, etoposide, teniposide, mitosis inhibitors, vinblastine, vincristine, vindesine, vinorelbine, paclitaxel, docetaxel, trofosfamide, chlorambucil, treosulfan, estramustine, nimustine, carmustine, lomustine, cytostatically active antibiotics such as dactinomycin, anthracycline, daunorubicin, doxorubicin (adriamycin), doxorubicin lipo, idarubicin, epirubicin, bleomycin, mitomycine, mitoxantrone, amsacrine, actinomycin D, hormones and hormone antagonists, buselerin, goselerin, leuprorelin, triptorelin, anti-estrogens, tamoxifen, aromatase inhibitors, aminoglutethimide, anastrozole, letrozole, exemestane, formestane, testolacton, glucocorticoides, miltefosine, hydroxy urea, tretinoin, topoisomerase inhibitors, camptothecin derivatives, irinotecan, hycamtin, topotecan, imatinib, amsacrine, pentostatin, bexaroten and asparaginase.

4. Combination according to claim 1, wherein the radio waves are in the frequency range from 3 to 30 MHz, preferably from 5 to 20 MHz and especially preferably from 10 to 15 MHz.

5. Combination according to claim 1, wherein the radio wave field formed by the radio waves is generated through capacitive coupling.

6. Combination according to claim 1, wherein the monoclonal antibodies, tyrosine-kinase inhibitors, angiogenesis inhibitors, farnesyl-transferase inhibitors, topoisomerase-I inhibitors, topoisomerase-II inhibitors, cytokines and/or antisense oligonucleotides are chosen from the group comprising cetuximab, trastuzumab, alemtuzumab, rituximab, bevacizumab, EMD7200, rencarex, HeFi-1, apolizumab, tositumomab, ABX-EGF, HuMAx-EGFR, labetuzumab, pemtumomab, triab, Mab17-1A (panorex), MDX-210, MDX-220, MDX-447, MDX-H210, gemtuzumab, ozogamicin, radio marked antibodies, Indium-111 (In-111) ibritumomab tiuxetan, yttrium-90 (Y-90) ibritumomab tiuxetan, Anti-CD80 mAb, WX-G250RIT (iodine-131), gefitinib, imatinib, PKI-166, CI-1033, SU-6668, cilengitide, neovastat, IM862, SU5415, SU5416, suramin, BMS214662, R115777 (zarnesta tipifarnib), aidesleukin, Interleukin-2, Interleukin-12, interferons, interferon-alpha 2a, interferon-alpha 2b, interferon beta, interferon gamma, tasonermin, AP12009.

7. Use of radio waves with at least one active agent chosen from the group
a) monoclonal antibodies and/or
b) tyrosine-kinase inhibitors and/or
c) angiogenesis inhibitors and/or
d) farnesyl-transferase inhibitors and/or
e) topoisomerase-I or -II inhibitors and/or
f) cytokines and/or
g) antisense oligonucleotides
for providing a combination for the prophylaxis and/or treatment of cancer, carcinogenic and/or benign tumors and metastases.

8. Use according to claim 7, wherein the cancer, the tumors and metastases are breast carcinomas, especially breast carcinomas recurrent after radiation and chemotherapy, skin carcinomas such as basaliomas, spinaliomas, melanomas, skin metastases, all malignant tumors which are located close to the body surface, soft tissue tumors, endometrial carcinoma, cervical carcinoma, ovarian cancer or respectively ovarian carcinoma, testicular carcinoma or respectively vulva carcinoma, germ cell tumors, prostate cancer, thyroid carcinomas, liver cell carcinomas, liver metastases, colon cancer or respectively colorectal carcinoma, rectal carcinoma, lung cancer such as small cell and non-small cell bronchial carcinoma, lymph node cancer, Hodgkin's and Non-Hodgkin's lymphomas, pancreas cancer or respectively pancreatic carcinoma, connective tissue tumor, soft tissue sarcoma, adenocarcinomas such as stomach, pancreas, gallbladder, esophagus carcinomas, other stomach carcinomas, osteolytic carcinomas and osteoplastic carcinomas, renal cell carcinomas, malignomas of the gastrointestinal tract, brain tumors such as glioblastomas, astrocytomas, tumors of the throat, nose and ear area, malignant neoplasma, neuroblastoma, choroidal melanoma, acute leukemia, acoustic neurinoma, ampullary carcinoma, anal carcinoma, bladder cancer, breast cancer, Burkift's lymphoma, corpus cancer, CUP-syndrome, cancer of the small intestine, tumors of the small intestine, ependymoma, epithelial cancer types, Ewing's tumors, gastrointestinal tumors, gallbladder cancer, uterine cancer, cervical cancer, gynecologic tumors, hematologic neoplasias, hairy cell leukemia, urethral cancer, skin cancer, brain metastases, hypophysis tumor, carcinoids, Kaposi's sarcoma, laryngeal cancer, bone cancer, colon carcinoma, craniopharyngiomas, cancer in the mouth area and on lips, leukemia, eyelid tumor, lymphomas, rectum cancer, medulloblastomas, melanomas, meningeomas, Hodkgin's disease, mycosis fungoides, nose cancer, neurinoma, renal cancer, oligodendroglioma, esophageal carcinoma, osteosarcomas, penile cancer, plasmocytoma, esophageal cancer, retinoblastoma, vaginal cancer, Schneeberger's disease, T-cell lymphoma (mycosis fungoides), thymoma, tube carcinoma, eye tumors, urethral cancer, urologic tumors, urothelial carcinoma, Wilm's tumor, and/or tongue cancer.

9. Use according to claim 7, wherein the radio waves are used simultaneously with the active agent or delayed by up to 72 hours, preferably 48 hours and especially preferably up to 24 hours.

10. Use according to claim 7, wherein the radio wave field generated by the radio waves is applied extra-tumorally.

11. Use of radio waves for increasing the activity of an active agent chosen from the group comprising
a) monoclonal antibodies and/or
b) tyrosine-kinase inhibitors and/or
c) angiogenesis inhibitors and/or
d) farnesyl-transferase inhibitors and/or
e) topoisomerase-I or -II inhibitors and/or
f) cytokines and/or
g) antisense oligonucleotides and/or
h) chemotherapeutics.

12. Use of radio waves for reducing the side effects of chemotherapeutics.

13. Use according to claim 11, wherein the chemotherapeutics are chosen from the group comprising: folic acid antagonists, methotrexate, antagonists of purine and pyrimidine bases, fluorouracil, capecitabine, gemcitabine, cytarabine, pentostatin, cytosine arabinoside, mercaptopurine, fludarabine, cladribine, thioguanine, paclitaxel, nitrourea, etoposide, campathecin, bleomycin, pliamycin, mitoxantron, cytochalasin B, gramicidin B, ethidiumbromide, emetin, mitomycin, tenoposides, colchicine, mithramycine, 1-Dehydrotestosterone, glucocorticoides, procaine tetracaine, lidocaine, propanolol, puromycine, streptozoticine, alkylating cytostatics, cyclophosphamide, ifosfamide, melphalan, thiotepa, busulfan, cisplatin, carboplatin, oxaliplatin, procarbazine, dacarbazine, temozolomide, etoposide, teniposide, mitosis inhibitors, vinblastine, vincristine, vindesine, vinorelbine, paclitaxel, docetaxel, trofosfamide, chlorambucil, treosulfan, estramustine, nimustine, carmustine, lomustine, cytostatically active antibiotics such as dactinomycin, anthracycline, daunorubicin, doxorubicin (adriamycin), doxorubicin lipo, idarubicin, epirubicin, bleomycin, mitomycine, mitoxantrone, amsacrine, actinomycin D, hormones and hormone antagonists, buselerin, goselerin, leuprorelin, triptorelin, anti-estrogens, tamoxifen, aromatase inhibitors, aminoglutethimide, anastrozole, letrozole, exemestane, formestane, testolacton, glucocorticoides, miltefosine, hydroxy urea, tretinoin, topoisomerase inhibitors, camptothecin derivatives, irinotecan, hycamtin, topotecan, imatinib, amsacrine, pentostatin, bexaroten and asparaginase.

14. Use according to claim 11, wherein the monoclonal antibodies, tyrosine-kinase inhibitors, angiogenesis inhibitors, farnesyl-transferase inhibitors, topoisomerase-I inhibitors, topoisomerase-II inhibitors, cytokines and/or antisense oligonucleotides are chosen from the group comprising cetuximab, trastuzumab, alemtuzumab, rituximab, bevacizumab, EMD7200, rencarex, HeFi-1, apolizumab, tositumomab, ABX-EGF, HuMAx-EGFR, labetuzumab, pemtumomab, triab, Mab17-1A (panorex), MDX-210, MDX-220, MDX-447, MDX-H210, gemtuzumab, ozogamicin, radio marked antibodies, indium-111 (In-111) ibritumomab tiuxetan, yttrium-90 (Y-90) ibritumomab tiuxetan, Anti-CD80 mAb, WX-G250RIT (iodine-131), gefitinib, imatinib, PKI-166, CI-1033, SU-6668, cilengitide, neovastat, IM862, SU5415, SU5416, suramin, BMS214662, R115777 (zarnesta tipifarnib), aldesleukin, Interleukin-2, Interleukin-12, interferons, interferon-alpha 2a, interferon-alpha 2b, interferon beta, interferon gamma, tasonermin, AP12009.

15. Use according to claim 11, wherein the radio waves are in the frequency range from 3 to 30 MHz, preferably from 5 to 20 MHz and especially preferably from 10 to 15 MHz.

16. Combination according to claim 3, wherein the radio waves are in the frequency range from 3 to 30 MHz, preferably from 5 to 20 MHz and especially preferably from 10 to 15 MHz.

17. Combination according to claim 3, wherein the radio wave field formed by the radio waves is generated through capacitive coupling.

18. Use according to claim 8, wherein the radio waves are used simultaneously with the active agent or delayed by up to 72 hours, preferably 48 hours and especially preferably up to 24 hours.

19. Use according to claim 8, wherein the radio wave field generated by the radio waves is applied extra-tumorally.

20. Use according to claim 13, wherein the radio waves are in the frequency range from 3 to 30 MHz, preferably from 5 to 20 MHz and especially preferably from 10 to 15 MHz.
